(19) United States
(12) Patent Application Publication
(10) Pub. No.: US 2023/0054234 A1
(43) Pub. Date: Feb. 23, 2023

(54) MULTI-METAL SALTS DISINFECTANT COMPOSITION

(71) Applicant: Onyx Lotus, LLC, Dover, DE (US)

(72) Inventor: Amyn Nanjee, Dover, DE (US)

(21) Appl. No.: 17/888,473

(22) Filed: Aug. 15, 2022

Related U.S. Application Data
(60) Provisional application No. 63/233,161, filed on Aug. 13, 2021.
(60) Provisional application No. 63/234,593, filed on Aug. 18, 2021.

(51) Int. Cl. A01N 59/20; A01N 59/16; A01N 25/22

(52) U.S. Cl. CPC A01N 59/20; A01N 59/16; A01N 25/22; A01P 1/00

(57) ABSTRACT

The present invention provides a disinfectant composition, method for preparing a disinfectant composition, and methods of disinfecting of a surface or article using the disinfectant composition. These compositions are light stable, non-toxic, and non-corrosive, achieve a greater than 99% kill rate on a variety of pathogens and do not contain nanoparticles.

### CROSS-REFERENCE TO RELATED APPLICATIONS

This application claims priority to U.S. Provisional Application 63/233,161 filed on Aug. 13, 2021 and U.S. Provisional Application 63/234,593 filed on Aug. 18, 2021, each of which is hereby incorporated by reference herein.

### FIELD OF THE INVENTION

The present invention generally relates to disinfectant compositions. Particularly, the present invention relates to disinfectant compositions comprising two or more metal ions. The disinfectant composition is shown to be effective as an antimicrobial agent, an antibacterial agent, an antifungal agent, an antiviral agent, or a combination thereof that is effective against many pathogens including the virus families including the SARS-CoV-2 virus and methods of using such disinfectant compositions.

### BACKGROUND OF THE INVENTION

Coronavirus disease 2019 (“COVID-19”) is caused by severe acute respiratory syndrome coronavirus 2 (“SARS-CoV-2”). Currently, no effective drug has been proven to treat SARS-CoV-2 infection in humans. The COVID-19 pandemic has led to millions of people being negatively affected globally. Intensive efforts are under way to gain more insight into the mechanisms of viral replication, in order to develop targeted antiviral therapies. However, development of medicines may take years.

The COVID-19 pandemic has emphasized the importance of environmental cleanliness and hygiene management involving a wide variety of surfaces. Despite the strict hygiene measures which have been enforced, it is has proven to be very difficult to sanitize surfaces all of the time. Even when sanitized, surfaces may get contaminated again.

Respiratory secretions or droplets expelled by infected individuals can contaminate surfaces and objects, creating fomites (contaminated surfaces). Viable SARS-CoV-2 virus can be found on contaminated surfaces for periods ranging from hours to many days, depending on the ambient environment (including temperature and humidity) and the type of surface. See, for example, Van Doremalen et al., “Aerosol and surface stability of SARS-CoV-2 as compared with SARS-CoV-1”, New England Journal of Medicine 2020; 382: 1564-1567; Pastorino et al., “Prolonged Infectivity of SARS-CoV-2 in Fomites”, Emerging Infectious Diseases 2020; 26(9); and Chin et al., “Stability of SARS-CoV-2 in different environmental conditions”, The Lancet Microbe, e10, Apr. 2, 2020.

There is consistent evidence of SARS-CoV-2 contamination of surfaces and the survival of the virus on certain surfaces. People who come into contact with potentially infectious surfaces often also have close contact with the infectious person, making the distinction between respiratory droplet and fomite transmission difficult to discern. However, fomite transmission is considered a feasible mode of transmission for SARS-CoV-2, given consistent findings about environmental contamination in the vicinity of infected cases and the fact that other coronaviruses and respiratory viruses can transmit this way (World Health Organization, “Transmission of SARS-CoV-2: implications for infection prevention precautions”, Jul. 9, 2020 via www.who.int). Virus transmission may also occur indirectly through touching surfaces in the immediate environment or objects contaminated with virus from an infected person, followed by touching the mouth, nose, or eyes. While use of face masks has, generally speaking, become widespread, use of hand gloves has not. Even with gloves, touching of mouth, nose, and eyes still frequently occurs, following the touch of a contaminated surface.

Therefore, there is a desire to prevent the transmission of pathogens (such as, but not limited to, SARS-CoV-2) via surfaces. One method of reducing pathogen transmission is to reduce the period of human vulnerability to infection by reducing the period of viability of SARS-CoV-2 on solids and surfaces.

Surfaces may be treated with chemical biocides, such as bleach and quaternary ammoniums salts, or UV light, to disinfect bacteria and destroy viruses within a matter of minutes. Biocides in liquids are capable of inactivating at least 99.99 wt % of SARS-CoV-2 in as little as 2 minutes, which is attributed to the rapid diffusion of the biocide to microbes and because water aids microbial dismemberment. However, these approaches cannot always occur in real-time after a surface is contaminated.

Alternatively, antimicrobial coatings may be applied to a surface in order to kill bacteria and/or destroy viruses as they deposit. However, to exceed 99.9 wt % reduction of bacteria and/or viruses, conventional antimicrobial coatings typically require at least 1 hour, a time scale which is longer than indirect human-to-human interaction time, such as in an aircraft or shared vehicles, for example. Existing solid coatings are limited by a low concentration of biocides at the surface due to slow biocide transport. The slow diffusion of biocides through the solid coating to the surface, competing with the removal of biocides from the surface by human and environmental contact, results in limited availability and requires up to 2 hours to kill 99.9 wt % of bacteria and/or deactivate 99.9 wt % of viruses.

Various alcohol-based disinfectants have been launched which are generally more effective against bacteria compared to viruses. These products are available in solution, gel, or spray form for use on human hand and body surfaces as well as on non-human surfaces such as wood, textiles, metals, polymers, etc. However, the efficacy of alcohol-based disinfectants against viruses has not been established.

Accordingly, what is needed is an efficient anti-viral composition for application to surfaces, or within bulk materials, in order to effectively prevent the spread of the SARS-CoV-2 virus and other viruses.

### DETAILED DESCRIPTION OF EMBODIMENTS OF THE INVENTION

The compositions and methods of the present invention will be described in detail by reference to various non-limiting embodiments.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention.

Reference throughout this specification to “one embodiment,” “some embodiments”, “certain embodiments,” “one or more embodiments,” “ one embodiment,” or “an embodiment” means that a particular feature, structure, material, or characteristic described in connection with the embodiment is included in at least one embodiment of the invention. Thus, the appearances of phrases containing the term “embodiment(s)” in various places throughout this specification are not necessarily referring to the same embodiment of the invention. Furthermore, the particular features, structures, materials, or characteristics may be combined in any suitable manner in one or more embodiments.

As used in this specification and the appended claims, the singular forms “a,” “an,” and “the” include plural referents unless the context clearly indicates otherwise.

Unless defined otherwise, all technical and scientific terms used herein have the same meaning as is commonly understood by one of ordinary skill in the art to which this invention belongs.

In the present disclosure, the percentage symbol % refers to mass percentage and weight percentage (wt %), unless otherwise stated.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms, except when used in a Markush group. Thus, in some embodiments not otherwise explicitly recited, any instance of “comprising” may be replaced by “consisting of” or, alternatively, by “consisting essentially of.”

Reference throughout this specification to one embodiment, certain embodiments, one or more embodiment or an embodiment means that a particular feature, structure, material, or characteristic described in connection with the embodiment is included in at least one embodiment of the invention. Thus, the appearance of phrases such as in one or more embodiments insert in embodiments, in one embodiment or in an embodiment in various places throughout this specification are not necessarily referring to the same embodiment of the invention. Furthermore, the features, structures, materials, or characteristics may be combined in any suitable manner in one or more embodiments. Also, all references herein to the “invention” shall mean embodiments of the invention.

As intended herein, “disinfectant” refers to a material capable of causing the inactivation of viruses (such as, but not limited to, SARS-CoV-2 virus), bacteria, yeasts, fungi, molds, or other microbes that may cause human infection.

In some variations, the present invention pertains to the synthesis of multi-metal salts to be used as disinfectants. In some variations, the present invention relates to the methodology of synthesis of multi-metal salts, providing a composition with disinfection properties against several viral and bacterial families. In this disclosure, a “multi-metal salt” is a salt that contains at least two atomically distinct metals.

It is an objective of the present invention to provide a method to synthesize a multi-metal ion salt that can act as an antimicrobial agent when applied to various surfaces and substrates, such as plastic, wood, and metal.

It is another objective of the present invention to provide disinfectant properties which are effective against different viruses including, but not limited to, SARS-CoV-2 virus that causes COVID-19.

It is a further objective of the present invention to provide disinfectant properties by virtue of synthesizing a salt consisting of two or more different metal ions.

It is further an objective of the present invention to provide a multi-metal salt to be used within a light-stable and heat-stable disinfectant composition.

The multi-metal salt may be combined with one or more other components, such as polymers, surfactants, reducing agents, complexing agents, chelating agents, other additives, or combinations thereof.

The disinfectant composition contains at least two different metals, at least one of which is an active component (active herein refers to disinfectant properties). In preferred embodiments, all metals contained in the disinfectant composition are active as a disinfectant.

The metals that may be utilized in the disinfectant composition include, but are not limited to, silver (Ag), copper (Cu), zinc (Zn), gold (Au), cobalt (Co), nickel (Ni), zirconium (Zr), molybdenum (Mo), alloys thereof, or combinations of the foregoing. The metals are preferably contained in the disinfectant composition as metal salts, rather than as pure metals or as solely metal ions.

In some embodiments, the disinfectant composition includes a silver salt.

In some embodiments, the disinfectant composition includes a copper salt.

In some embodiments, the disinfectant composition includes a zinc salt.

In certain embodiments, the disinfectant composition includes a silver salt as well as a copper salt.

In certain embodiments, the disinfectant composition includes a silver salt as well as a zinc salt.

In certain embodiments, the disinfectant composition includes a copper salt as well as a zinc salt.

In certain embodiments, the disinfectant composition includes a silver salt, a copper salt, and a zinc salt.

The total concentration of all metal salts in the disinfectant composition may vary, such as from about 0.00001 wt % to about 100 wt %, preferably from about 0.01 wt % to about 50 wt %, or from about 0.1 wt % to about 25 wt %. In various embodiments, the total metal-salt concentration is about, at least about, or at most about 0.00001 wt %, 0.0001 wt %, 0.001 wt %. 0.01 wt %, 0.1 wt %, 0.5 wt %, 1 wt %, 1.5 wt %, 2 wt %, 3 wt %, 4 wt %, 5 wt %, 10 wt %, 15 wt %, 20 wt %, 25 wt %, 30 wt %, 40 wt %, 50 wt %, 60 wt %, 70 wt %, 80 wt %, or 90 wt %, including all intervening ranges.

In this specification, reference to “intervening ranges” is in reference to embodiments in which there is a sub-selection of numbers within a larger range of numbers. For instance, the total metal-salt concentration may specifically be sub-selected within a range of 0.01-5.0 wt %, 1.0-3.0 wt %, or any other range that starts and ends with two of the recited concentrations.

The individual concentrations of the different metal salts may be the same or different.

In various embodiments, a silver salt is present in a concentration from about 0.001 wt % to about 25 wt %, such as from about 0.01 wt % to about 10 wt %, or from about 0.1 wt % to about 5 wt %.

The silver salt (when present) may be any compound of the form AgₙXₘ (n>0, m>0) that is capable of releasing silver cations, usually as Ag⁺ but potentially as Ag²⁺, Ag³⁺, etc., in addition to Ag⁺ or instead of Ag⁺. For convenience in the rest of this specification, reference will be made to monocationic Ag⁺ with the understanding that other silver ions may be released. The species X may be a single atom such as chlorine (Cl) or may itself contain multiple atomic species, such as a nitrate group (NO₃).

Exemplary silver salts include silver halides such as silver chloride (AgCl), silver fluoride (AgF, AgF₂, AgF₃, and/or Ag₂F), silver bromide (AgBr), silver iodide (AgI), or a combination thereof. Other exemplary silver salts include silver nitrate (AgNO₃), silver acetate (AgCH₃COO), silver carbonate (Ag₂CO₃), silver citrate (Ag₃C₆H₅O₇), silver lactate (AgC₃H₅O₃), silver phosphate (Ag₃PO₄), silver sulfate (Ag₂SO₄), silver perchlorate (AgClO₄), silver trifluoroacetate (AgCF₃COO), silver sulfadiazine (AgC₁₀H₉N₄O₂S), and combinations thereof, for example. In some preferred embodiments, the silver salt is silver nitrate. Exemplary silver-containing compounds that are not ordinarily classified as salts (but for this disclosure, are regarded as salts) include, but are not limited to, silver sulfide (Ag₂S), silver oxide (Ag₂O), silver nitride (Ag₃N), silver hydride (AgH), and silver carbide (Ag₂C₂, usually referred to as silver acetylide).

The particle size of the silver salt may vary. In some embodiments, the average particle size of the silver salt is selected from about 0.1 microns to about 10 microns. In various embodiments, the average particle size of the silver salt is about, at least about, or at most about 0.2, 0.3, 0.4, 0.5, 0.6, 0.7, 0.8, 0.9, 1, 1.5, 2, 2.5, 3, 4, 5, 6, 7, 8, 9, or 10 microns, including all intervening ranges. In certain embodiments, the average particle size of the silver salt is at least about 0.5 microns (500 nanometers). Note that it is possible to utilize silver salt particles having an average particle size less than 0.1 microns, such as about 90, 80, 70, 60, 50, 40, 30, 20, or 10 nanometers or even smaller (i.e., nanoparticles). Typically, however, the average particle size of the silver salt is greater than 0.1 micron (100 nanometers). Also, it is possible to utilize silver salt particles having an average particle size larger than 10 microns, such as about 20, 30, 40, 50, 60, 70, 80, 90, or 100 microns or even larger.

When the silver salt is chemically or physically bound to other components (such as a polymer or a chelating agent) to form a complexed particle, the measured particle size will typically be that of the complexed particle.

Particle sizes may be measured by a variety of techniques, including dynamic light scattering, laser diffraction, image analysis, or sieve separation, for example. Dynamic light scattering is a non-invasive, well-established technique for measuring the size and size distribution of particles typically in the submicron region, and with the latest technology down to 1 nanometer. Laser diffraction is a widely used particle-sizing technique for materials ranging from hundreds of nanometers up to several millimeters in size. Exemplary dynamic light scattering instruments and laser diffraction instruments for measuring particle sizes are available from Malvern Instruments Ltd., Worcestershire, UK. Image analysis to estimate particle sizes and distributions can be done directly on photomicrographs, scanning electron micrographs, or other images. Finally, sieving is a conventional technique of separating particles by size.

The particle shape of the silver salt may vary. For example, the particle shape may be selected from spheres, ovoids, cubes, pyramids, plates, rods, needles, random shapes, or a combination thereof. The silver salt may be characterized by an average aspect ratio of the maximum length scale to the minimum length scale. The average aspect ratio may vary from 1 (e.g., spheres or cubes) to 100 or greater (e.g., needle-like particles). In some embodiments, substantially a single particle shape characterizes the silver salt. In other embodiments, a combination of multiple particle shapes characterizes the silver salt within the composition. Particle shape may be determined using image analysis with photomicrographs, scanning electron micrographs, or other images.

In various embodiments, a copper salt is present in a concentration from about 0.001 wt % to about 25 wt %, such as from about 0.01 wt % to about 10 wt %, or from about 0.1 wt % to about 5 wt %.

Copper, like silver, has known antiviral properties. For example, it has been shown that copper ions, like silver ions, have specific affinity for double-stranded DNA. See, for example, Lu et al., “Silver nanoparticles inhibit hepatitis B virus replication”, Antiviral Therapy 2008, 13, 253-62 and Borkow et al., “Copper as a biocidal tool”, Current Medicinal Chemistry, 2005, 12, 2163-75, which are hereby incorporated by reference herein.

The copper salt (when present) may be any compound of the form CuₚY_{q} (p>0, q>0) that is capable of releasing copper cations, usually as Cu²⁺ but potentially as Cu⁺, Cu³⁺, etc., in addition to Cu²⁺ or instead of Cu²⁺. The species Y may be a single atom such as chlorine (Cl) or may itself contain multiple atomic species, such as a nitrate group (NO₃). In this disclosure, Y does not refer to the element yttrium.

Exemplary copper salts include copper halides such as copper chloride (CuCl and/or CuCl₂), copper fluoride (CuF and/or CuF₂), copper bromide (CuBr and/or CuBr₂), copper iodide (CuI), or a combination thereof. Other exemplary copper salts include copper nitrate (Cu(NO₃)₂), copper acetate (Ag(CH₃COO)₂), copper carbonate (CuCO₃), and copper sulfate (CuSO₄), for example. In some preferred embodiments, the copper salt is copper nitrate. Exemplary copper-containing compounds that are not ordinarily classified as salts (but for this disclosure, are regarded as salts) include, but are not limited to, copper sulfide (e.g., CuS), copper oxide (CuO and/or Cu2O), copper nitride (Cu₃N₂), copper hydride (CuH), and copper carbide (Cu₂C_{2,} usually referred to as copper acetylide).

In various embodiments, a zinc salt is present in a concentration from about 0.001 wt % to about 25 wt %, such as from about 0.01 wt % to about 10 wt %, or from about 0.1 wt % to about 5 wt %.

Zinc, like silver and copper, has known antiviral properties. See, for example, Read et al., “The Role of Zinc in Antiviral Immunity”, Adv Nutr 2019;10, 696-710, which is hereby incorporated by reference herein. The zinc salt (when present) may be any compound of the form ZnᵤQᵥ (u>0, v>0) that is capable of releasing zinc cations, usually as Zn²⁺ but potentially as Zn⁺, Zn³⁺, etc., in addition to Zn²⁺ or instead of Zn²⁺. The species Q may be a single atom such as chlorine (Cl) or may itself contain multiple atomic species, such as a nitrate group (NO₃).

Exemplary zinc salts include zinc halides such as zinc chloride (ZnCl₂), zinc fluoride (ZnF₂), zinc bromide (ZnBr₂), zinc iodide (ZnI₂), or a combination thereof. Other exemplary zinc salts include zinc nitrate (Zn(NO₃)₂), zinc acetate (Zn(CH₃COO)₂), zinc carbonate (ZnCO₃), and zinc sulfate (ZnSO₄), for example. In some preferred embodiments, the zinc salt is zinc nitrate. Exemplary zinc-containing compounds that are not ordinarily classified as salts (but for this disclosure, are regarded as salts) include, but are not limited to, zinc sulfide (e.g., ZnS), zinc oxide (ZnO), zinc nitride (Zn₃N₂), zinc hydride (ZnH₂), and zinc carbide (ZnC).

When silver salts, copper salts, and/or zinc salts are employed in a disinfectant composition, such salts and their concentrations may be independently selected from the above lists, for example.

In some embodiments employing both silver salts and copper salts, the counterions or bonded species X and Y, within AgₙXₘ and CuₚY_{q}, respectively, may be the same (X=Y), or they may be different (X≠Y). For example, a combination of silver nitrate and copper nitrate may be employed (X=Y), or a combination of silver chloride and copper nitride may be employed (X≠Y).

In some embodiments employing both silver salts and zinc salts, the counterions or bonded species X and Q, within AgₙXₘ and ZnᵤQᵥ, respectively, may be the same (X=Q), or they may be different (X≠Q). For example, a combination of silver nitrate and copper nitrate may be employed (X=Y), or a combination of silver chloride and zinc hydride may be employed (X≠Y).

In some embodiments employing both copper salts and zinc salts, the counterions or bonded species Y and Q, within CuₚX_{q} and ZnᵤQᵥ, respectively, may be the same (Y=Q), or they may be different (Y≠Q). For example, a combination of copper nitrate and zinc nitrate may be employed (X=Y), or a combination of copper fluoride and zinc acetate may be employed (X≠Y).

The different metal salts are not typically chemically bound to each other, although some degree of association may occur. For example, ion-exchange reactions between a silver salt and a copper salt may take place such that counterions or bonded species X and Y, within AgₙXₘ and CuₚY_{q}, respectively, may switch.

The particle size of a copper salt (when present) may vary. In some embodiments, the average particle size of the copper salt is selected from about 0.1 microns to about 10 microns. In various embodiments, the average particle size of the copper salt is about, at least about, or at most about 0.2, 0.3, 0.4, 0.5, 0.6, 0.7, 0.8, 0.9, 1, 1.5, 2, 2.5, 3, 4, 5, 6, 7, 8, 9, or 10 microns, including all intervening ranges. In certain embodiments, the average particle size of the copper salt is at least about 0.5 microns (500 nanometers). Typically, the average particle size of the copper salt is greater than 0.1 micron (100 nanometers). Also it is possible to utilize copper salt particles having an average particle size larger than 10 microns, such as about 20, 30, 40, 50, 60, 70, 80, 90, or 100 microns or even larger. When the copper salt is chemically or physically bound to other components (such as a polymer, a reducing agent, or another metal salt) to form a complexed particle, the measured particle size will typically be that of the complexed particle.

The particle size of a zinc salt (when present) may vary. In some embodiments, the average particle size of the zinc salt is selected from about 0.1 microns to about 10 microns. In various embodiments, the average particle size of the zinc salt is about, at least about, or at most about 0.2, 0.3, 0.4, 0.5, 0.6, 0.7, 0.8, 0.9, 1, 1.5, 2, 2.5, 3, 4, 5, 6, 7, 8, 9, or 10 microns, including all intervening ranges. In certain embodiments, the average particle size of the zinc salt is at least about 0.5 microns (500 nanometers). Typically, the average particle size of the zinc salt is greater than 0.1 micron (100 nanometers). Also it is possible to utilize zinc salt particles having an average particle size larger than 10 microns, such as about 20, 30, 40, 50, 60, 70, 80, 90, or 100 microns or even larger. When the zinc salt is chemically or physically bound to other components (such as a polymer, a reducing agent, or another metal salt) to form a complexed particle, the measured particle size will typically be that of the complexed particle.

The average particle sizes of the different metal salts may be the same or different. For example, in a composition that includes a silver salt, a copper salt, and a zinc salt, the average silver-salt particle size, the average copper-salt particle size, and the average zinc-salt particle size may all be approximately the same, or two of them may be about the same and the other salt larger or smaller, or all three may be different in size.

The particle shape of the copper salt (when present) and/or the zinc salt (when present) may vary, similar to the silver salt particle shape discussed earlier. For example, the particle shape may be selected from spheres, ovoids, cubes, pyramids, plates, rods, needles, random shapes, or a combination thereof. The salt may be characterized by an average aspect ratio of the maximum length scale to the minimum length scale. The average aspect ratio may vary from 1 (e.g., spheres or cubes) to 100 or greater (e.g., needle-like particles). In some embodiments, substantially a single particle shape characterizes the copper salt and/or zinc salt. In other embodiments, a combination of multiple particle shapes characterizes the copper salt and/or zinc salt within the composition. Particle shape may be determined using image analysis with photomicrographs, scanning electron micrographs, or other images.

In embodiments employing a silver salt, a copper salt, and a zinc salt, the particle shape(s) of the different salt particle shapes may be the same or different.

Processes to produce the disinfection composition will now be described, without limitation.

Before describing several exemplary embodiments of the process, it is to be understood that the invention is not limited to the details of the process or process steps set forth in the following description. The invention is capable of other embodiments and of being practiced or being carried out in various other ways.

In some variations, an electrochemical cell is utilized to produce a multi-metal salt, as follows. The electrochemical cell contains a bath of an electrolyte solution, as well as at least two electrodes. At least one electrode is an anode, and at least one electrode is a cathode. There may be multiple anodes and/or multiple cathodes. A third electrode may be a reference electrode or a reservoir electrode, for example. Each electrode may contain one metal or more than one metal, such as a metal alloy.

The electrolyte solution contain one or more precursors to the desired multi-metal salts. For example, if the multi-metal salt is a metal nitrate (e.g., silver nitrate and copper nitrate), then the electrolyte solution preferably contains nitric acid or a salt thereof (e.g., sodium nitrate).

The electrolyte solution may also contain any desired additives, such as a chelating agent. In some embodiments, a chelating agent (discussed later in the specification) is included in the electrolyte solution. The chelating agent may be an organic acid (e.g., citric acid) that may itself provide electrolytic function to the electrolyte solution, by generating ions that can assist in the electrochemical reactions taking place.

There is typically one cathode and multiple anodes, wherein each anode is selected to contain the desired metals in the final multi-metal salt. For example, if the multi-metal salt contains silver, copper, and zinc, there may be three anodes that separately contain silver, copper, and zinc. It is also possible to employ a single anode having a metal alloy containing multiple metals—such as a metal alloy comprising silver, copper, and zinc. Or one anode may contain a relatively pure metal (e.g., silver) while another anode contains a metal alloy, such as one containing copper and zinc (e.g., brass). An anode may generally contain one, two, three, four, five, or more metals. (Note: When there are multiple, physically distinct anodes with different compositions, the collection of anodes may be referred to as “the anode” if desired.)

Synthesis of the multi-metal salt is typically achieved in the electrochemical cell by passing an electrical current between the cathode and anode, with an applied voltage. The applied voltage enables current to flow between the electrodes, using suitable current collectors that are connected to the electrodes and to an external circuit via electrical leads. The electrochemical potential that arises from the applied voltage causes the multi-metal salt to be generated within the electrolyte solution (liquid phase). Preferably, multi-metal salts are not generated on electrode surfaces, or if they are, only transiently followed by diffusion into the liquid bath.

The applied voltage will generally be dictated by the electrochemical reactions to be carried out, which will in turn be based on the metal salts being produced. Typically, the range of voltage applied during synthesis is 0.01 V to 1000 V, such as from about 0.1 V to about 240 V, or from about 0.5 V to about 5 V. In various embodiments, the applied voltage is about, at least about, or at most about 0.01 V, 0.05 V, 0.1 V, 0.2 V, 0.3 V, 0.4 V, 0.5 V, 0.6 V, 0.7 V, 0.8 V, 0.9 V, 1 V, 1.5 V, 2 V, 2.5 V, 3 V, 4 V,5 V, 6 V, 7 V, 8 V, 9 V, 10 V, 20 V, 30 V, 40 V, 50 V, 75 V, 100 V, 150 V, 200 V, or 240 V, including any intervening ranges. In certain situations, an applied voltage is not necessary as the intended chemical reactions proceed to some extent. However, in those situations, usually the reaction rate is exceeding slow or the reaction conversion is too low.

The current that flows through the external circuit, under the applied voltage, may vary widely and will be dictated by the electrochemical reactions to be carried out as well as the size of the system. Whereas the voltage is an intrinsic property that does not depend on the system capacity, the current is an extrinsic property—more electrons must flow as the overall system becomes bigger. The current that flows through the external circuit may be direct current or alternating current.

The synthesis of the multi-metal salt may be conducted in a batch process, a semi-batch process, a semi-continuous process, or continuous process.

In a batch process, the electrolyte solution is stagnant. The electrochemical reactions that take place under the applied voltage are allowed to proceed for an effective period of time. The multi-metal salt forms in the solution. The multi-metal salt may precipitate out of the electrolyte solution and then may be recovered, such as by decantation, filtration, or evaporation. The multi-metal salt may remain suspended in the electrolyte solution, but not fully precipitate, in which case the multi-metal salt may be recovered, such as by filtration or centrifugation. Alternatively, or additionally, the multi-metal salt may remain dissolved in the electrolyte solution, in which case the electrolyte solution may be recovered and then treated, such as by adjusting temperature, pH, or using another solvent, to recover the multi-metal salt.

In a continuous process, the electrolyte solution continuously flows through the electrochemical cell. In some embodiments, fresh electrolyte solution enters the electrochemical cell which is equipped with electrodes as described above. The multi-metal salt is continuously produced in the reactor. The reactor may be configured such that the multi-metal salt is continuously recovered, such as by using an in-line filter at an exit of the reactor. In some embodiments, an electrolyte solution containing dissolved, suspended, or precipitated metal salts (or a combination thereof) is recovered from the reactor. Then, the multi-metal salt may be recovered, such as by decantation, filtration, evaporation, centrifugation, or other means, including potentially adjusting temperature or pH, or by using another solvent, to recover the multi-metal salt. In certain embodiments of a continuous process, there is slow outflow and inflow of the electrolyte solution, thereby preventing saturation of metal salt in the vessel.

A semi-batch or semi-continuous process is a process that has attributes of a batch process as well as attributes of a continuous process. For example, in certain embodiments, there is intermittent inflow of liquid electrolyte and/or intermittent outflow of liquid electrolyte that contains the multi-metal salt.

The electrochemical cell may be contained within a reaction vessel (also referred to herein as a reactor). The reactor may be equipped with agitation for improved mass transfer. In a continuous process, there will typically be some amount of agitation due to the dynamics of the inflow and outflow. Nevertheless, in some embodiments whether batch or continuous, agitation may be achieved using impellers, rotating vessels, sonication, or other means. The reactor may be operated at a range of temperatures, pressures, and residence times.

In certain embodiments, the electrolyte solution contains a chelating agent (e.g., citric acid) that is part of the final disinfectant composition, i.e., remains bound to the multi-metal salt after it is recovered from the process. Chelating agents are discussed in more detail below.

In some embodiments, the disinfectant composition includes a polymer, such as a hydrophilic polymer. The polymer may be selected from the group consisting of polyacrylamide, poly(acrylamide-co-acrylic acid), poly(vinyl alcohol), poly(vinyl pyrrolidone), poly(ethylene oxide), carboxy methylcellulose, and combinations thereof.

The polymer, when included, may be present in a concentration from about 0.1 wt % to about 75 wt % within the disinfectant composition. In some embodiments, the polymer is present in a concentration from about 1 wt % to about 10 wt %, or from about 1 wt % to about 5 wt %. In various embodiments, the polymer is present in a concentration of about, at least about, or at most about 0.1 wt %, 0.2 wt %, 0.3 wt %, 0.4 wt %, 0.5 wt %, 1 wt %, 1.5 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, or 75 wt %, including all intervening ranges.

In some embodiments, the disinfectant composition includes a reducing agent. A reducing agent is a chemical that is capable of reducing a cation to cause an acceptance of one or more electrons (donated by the reducing agent), decreasing the cation charge to a less-positive charge, to a neutral molecule, or to a negatively charged anion. The reducing agent may also be referred to as a complexing agent.

In some embodiments, the reducing agent is selected from the group consisting of citric acid, citrate salt, ascorbic acid, ascorbate salt, ethylenediaminetetraacetic acid (EDTA), ethylenediaminetetraacetate salt, and combinations thereof. Exemplary citrate salts include sodium citrate (also referred to as trisodium citrate), potassium citrate, potassium-sodium citrate, and potassium-magnesium citrate, for example. Exemplary ascorbate salts include sodium ascorbate, calcium ascorbate, and potassium ascorbate, for example. Exemplary ethylenediaminetetraacetate salts include disodium ethylenediaminetetraacetate, dipotassium ethylenediaminetetraacetate, sodium calcium ethylenediaminetetraacetate, tetrasodium ethylenediaminetetraacetate, or a combination thereof. Generally, EDTA salts may include ammonium, calcium, copper, iron, potassium, manganese, sodium, or zinc salts of EDTA. Other organic acids, organic-acid salts, aminopolycarboxylic acids, or aminopolycarboxylate salts may be employed as reducing agents.

For example, a reducing agent may be an organic compound selected from the group consisting of formic acid, glyoxilic acid, oxalic acid, acetic acid, glocolic acid, acrylic acid, pyruvic acid, malonic acid, propanoic acid, hydroxypropanoic acid, lactic acid, glyceric acid, fumaric acid, maleic acid, oxaloacetic acid, crotonoic acid, acetoacetic acid, 2-oxobutanoic acid, methylmalonic acid, succinic acid, methylsuccinic acid, malic acid, tartaric acid, dihydroxytartaric acid, butanoic acid, hydroxybutanoic acid, itaconic acid, mesaconic acid, oxoglutaric acid, glutaric acid, valeric acid, pivalic acid, aconitic acid, ascorbic acid, citric acid, isocitric acid, adipic acid, caproic acid, benzoic acid, salicylic acid, gentisic acid, protocatechuic acid, gallic acid, cyclohexanecarboxylic acid, pimelic acid, phthalic acid, terephthalic acid, phenylacetic acid, toluic acid, mandelic acid, suberic acid, octanoic acid, cinnamic acid, nonanoic acid, salts thereof, and combinations of the foregoing.

The reducing agent, when included, may be present in a concentration from about 0.1 wt % to about 50 wt %, for example. In various embodiments, the concentration of the reducing is about, at least about, or at most about 0.1 wt %, 0.2 wt %, 0.3 wt %, 0.4 wt %, 0.5 wt %, 1 wt %, 1.5 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, or 50 wt %, including all intervening ranges.

In some embodiments, the disinfectant composition includes a chelating agent. A chelating agent is capable of binding to at least one of the metals present in the composition. By binding to a metal (e.g., Ag), the chelating agent ensures that the metal remains bound in the metal salt (e.g., AgNO₃) rather than being oxidized from bound Ag to free Ag⁺ ions. A chelating agent may also be a reducing agent, but not necessarily. Preferred chelating agents are organic acids, such as citric acid, ascorbic acid, malic acid, fumaric acid, tartaric acid, ethylenediaminetetraacetic acid, salts thereof, or a combination of the foregoing, for example. In some embodiments, a chelating agent may be an inorganic acid, such as phosphoric acid.

In some embodiments, the chelating agent is selected from the group consisting of citric acid, a citrate salt, ascorbic acid, an ascorbate salt, ethylenediaminetetraacetic acid (EDTA), an ethylenediaminetetraacetate salt, and combinations thereof. Exemplary citrate salts include sodium citrate (also referred to as trisodium citrate), potassium citrate, potassium-sodium citrate, diammonium citrate, and potassium-magnesium citrate, for example. Exemplary ascorbate salts include sodium ascorbate, calcium ascorbate, ammonium ascorbate and potassium ascorbate, for example. Exemplary ethylenediaminetetraacetate salts include disodium ethylenediaminetetraacetate, diammonium ethylenediaminetetraacetate, dipotassium ethylenediaminetetraacetate, sodium calcium ethylenediaminetetraacetate, tetrasodium ethylenediaminetetraacetate, or a combination thereof. Generally, EDTA salts may include ammonium, calcium, copper, iron, potassium, manganese, sodium, or zinc salts of EDTA. Other organic acids, organic-acid salts, aminopolycarboxylic acids, or aminopolycarboxylate salts may be employed as chelating agents.

For example, a chelating agent may be an organic compound selected from the group consisting of formic acid, glyoxilic acid, oxalic acid, acetic acid, glocolic acid, acrylic acid, pyruvic acid, malonic acid, propanoic acid, hydroxypropanoic acid, lactic acid, glyceric acid, fumaric acid, maleic acid, oxaloacetic acid, crotonoic acid, acetoacetic acid, 2-oxobutanoic acid, methylmalonic acid, succinic acid, methylsuccinic acid, malic acid, tartaric acid, dihydroxytartaric acid, butanoic acid, hydroxybutanoic acid, itaconic acid, mesaconic acid, oxoglutaric acid, glutaric acid, valeric acid, pivalic acid, aconitic acid, ascorbic acid, citric acid, isocitric acid, adipic acid, caproic acid, benzoic acid, salicylic acid, gentisic acid, protocatechuic acid, gallic acid, cyclohexanecarboxylic acid, pimelic acid, phthalic acid, terephthalic acid, phenylacetic acid, toluic acid, mandelic acid, suberic acid, octanoic acid, cinnamic acid, nonanoic acid, salts thereof, and combinations of the foregoing.

The chelating agent may be present in a concentration from about 0.1 wt % to about 50 wt %, such as from about 1 wt % to about 25 wt %, for example. In various embodiments, the concentration of the reducing is about, at least about, or at most about 0.1 wt %, 0.2 wt %, 0.3 wt %, 0.4 wt %, 0.5 wt %, 1 wt %, 1.5 wt %, 2 wt %, 3 wt %, 4 wt %, 5 wt %, 6 wt %, 7 wt %, 8 wt %, 9 wt %, 10 wt %, 15 wt %, 20 wt %, or 25 wt %, including all intervening ranges.

The disinfectant composition may further comprise a wetting agent. The wetting agent may function as a surfactant at interfaces between different components of the disinfectant composition. Alternatively, or additionally, the wetting agent may function as a surfactant at an interface between the disinfectant composition and a substrate surface to which the disinfectant composition is to be applied. Surfactants may be anionic, cationic, zwitterionic, or non-ionic surfactants.

In some embodiments, the wetting agent (when present) is selected from the group consisting of polyethoxylated castor oil; polypropylene glycol-polyethylene glycol block copolymers; polyoxyethylene sorbitan monooleate; sodium lauryl sulfate; sodium carboxymethyl cellulose; calcium carboxymethyl cellulose; hydrogenated or non-hydrogenated glycerolipids; ethoxylated or non-ethoxylated, linear or branched, saturated or monounsaturated or polyunsaturated C₆ to C₃₀ fatty acids or salts thereof; cyclodextrin; alkaline earth metal or amine salt ethoxylated or non-ethoxylated esters of sucrose; sorbitol; mannitol; glycerol or polyglycerol containing from 2 to 20 glycerol units; glycols combined with fatty acids, monoglycerides, diglycerides, triglycerides, or mixtures of glycerides of fatty acids; ethoxylated or non-ethoxylated, linear or branched, saturated or monounsaturated or polyunsaturated C₆ to C₃₀ fatty alcohols; sterols; cholesterol or derivatives thereof; ethoxylated or non-ethoxylated ethers of sucrose, sorbitol, mannitol, glycerol, or polyglycerol containing from 2 to 20 glycerol units; hydrogenated or non-hydrogenated, polyethoxylated vegetable oils; polyethylene glycol hydroxystearate; sphingolipids or sphingosine derivatives; polyalkyl glucosides; ceramides; polyethylene glycol-alkyl glycol copolymers; polyethylene glycol-polyalkylene glycol ether di-block or tri-block copolymers; diacetylated monoglycerides; diethylene glycol monostearate; ethylene glycol monostearate; glyceryl monooleate; glyceryl monostearate; propylene glycol monostearate; polyethylene glycol stearate; polyethylene glycol ethers; polyethylene glycol hexadecyl ether; polyethylene glycol monododecyl ether; polyethylene glycol nonyl phenyl ethers; polyethylene glycol octyl phenyl ethers; octylphenoxy polyethoxyethanol; polyhydroxyethyl-tert-octylphenolformaldehyde; poloxamers; polysorbates; sorbitan monolaurate; sorbitan monooleate; sorbitan monopalmitate, sorbitan monostearate; sorbitan sesquioleate; sorbitan trioleate; sorbitan tristearate; phospholipids; and combinations thereof.

In certain embodiments, the wetting agent is a surfactant selected from Kolliphor EL, Poloxamer 407, Tween 80, or Triton X-100. In certain embodiments, the wetting agent is selected from the group consisting of macrogol stearate 400, macrogol stearate 2000, polyoxyethylene 50 stearate, macrogol ethers, cetomacrogol 1000, lauramacrogols, nonoxinols, octoxinols, tyloxapol, poloxamers, polysorbate 20, polysorbate 40, polysorbate 60, polysorbate 65, polysorbate 80, polysorbate 85, and combinations thereof.

The wetting agent, when included, may be present in a concentration from about 0.01 wt % to about 5 wt %, for example. In various embodiments, the wetting agent is in a concentration of about, at least about, or at most about 0 wt %, 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, or 5 wt %, including all intervening ranges.

The disinfectant composition may further comprise a binding agent. In some embodiments, the binding agent is selected from the group consisting of melamine, thiols, fatty acids, adhesives, polymers, acrylates, and combinations thereof. The binding agent may also be referred to as a surface binder.

The binding agent, when included, may be present in a concentration from about 0.01 wt % to about 5 wt %, for example. In various embodiments, the binding agent is in a concentration of about, at least about, or at most about 0 wt %, 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, or 5 wt %, including all intervening ranges.

The disinfectant composition may include a liquid solvent. The liquid solvent dissolves at least some of the composition components, and preferably dissolves all of the composition components, at least to some extent (and preferably, substantially completely). A typical solvent is water. Other polar solvents may be employed. Polar solvents may be protic polar solvents or aprotic polar solvents. Exemplary polar solvents include, but are not limited to, water, alcohols, ethers, esters, ketones, aldehydes, carbonates, and combinations thereof. The liquid solvent may include, or consist essentially of, an electrolyte.

The choice of solvent will generally be dictated primarily by the selection of metal salts. The solvent may also be chosen based, to some extent, on the selection of the chelating agent and/or optional components, if present. For example, when the silver salt is silver nitrate, water is an effective solvent because silver nitrate is highly soluble in water. An additive may be used to increase the water solubility of a metal salt.

The concentration of solvent may vary. The solvent concentration may be the minimum concentration that dissolves the silver-containing compound, or may be present in excess (which is typical). For example, the solvent concentration may be selected from about 10 wt % to about 99 wt %, such as about, at least about, or at most about 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, or 99 wt %, including all intervening ranges.

While the disinfectant composition is typically prepared with a solvent, it is noted that a dried form of the disinfectant composition may be prepared, such as in powder form. Spray drying may be used for making a powder form of the disinfectant composition. A disinfectant composition may be completely dry (i.e., no water present) or may contain some water but less water than necessary for equilibrium dissolution of all components, or less water than necessary for equilibrium dissolution of the metal salts. The dry disinfectant composition may be packaged, stored, sold, etc. and a solvent (e.g., water) then added at a later time, such as prior to or during use.

The pH of the disinfectant composition is preferably selected from about 5 to about 9, more preferably from about 6 to about 8, and most preferably from about 6.5 to about 7.5 (e.g., about 7). Some embodiments provide a slightly basic disinfectant composition, with a pH from about 7 to about 10, such as from about 7 to about 9, or from about 7 to about 8. Optionally, a weak base is added to the disinfectant composition in order to maintain slight basicity. In various embodiments, the pH of the disinfectant composition is about, at least about, or at most about 5, 5.5, 6.0, 6.1, 6.2, 6.3, 6.4, 6.5, 6.6, 6.7, 6.8, 6.9, 6.95, 7.0, 7.05, 7.1, 7.2, 7.3, 7.4, 7.5, 7.6, 7.7, 7.8, 7.9, 8.0, 8.5, or 9.0, including all intervening ranges. A pH buffer may be included in the disinfectant composition to help stabilize its pH.

The disinfectant composition exhibits antimicrobial properties, antibacterial properties, antiviral properties, antifungal properties, or a combination thereof against a variety of pathogens as verified by the following tests: for bacteria and fungi: AOAC Use Dilution Method (UDM), ASTM E 2315, ISO 22196:2011; and for viruses: AATCC 100-20124, IS018184:2019, ISO 21702:2019, Rt-PCR, liquid-liquid contact. For viruses, the Fonsum Pharma test against a Covid-19 RT-PCR Evaluation was utilized. The pathogen kill rate is greater than 99% after less than a 5-minute period of time and pathogenic sterility is maintained for up to 60 days. In some embodiments, pathogenic sterility is maintained on a surface for up to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

Generally speaking, the present invention is not limited by any particular hypothesis or mechanism of action of metal ions in inactivating viruses (e.g., SARS-CoV-2 virus), bacteria, fungi, or yeasts. For example, metal ions may cause lipid peroxidation of a viral or bacterial membrane via formation of reactive species, causing cellular lysis. Adhesion or interaction of metal ions to membrane saccharides, lipids, or proteins may cause membrane deformation, leading to loss of membrane potential and inactivation. Direct biocidal effects of metal ions may occur through interactions with DNA or critical cellular proteins. Silver is a photosensitizer which generates singlet oxygen when exposed to light. The singlet oxygen oxidizes the viral or bacterial protein and/or lipid, consequently leading to the inactivation of microbes. Combinations of multiple mechanisms are possible.

The disinfectant composition may contain various additives, in addition to the primary and optional components described above. A wide variety of additives may be incorporated, such as (but not limited to) diluents, carriers, vehicles, excipients, fillers, viscosity-modifying agents (e.g., thickeners or thinners), UV stabilizers, thermal stabilizers, antioxidants, pH buffers, acids, bases, metals (e.g., neutral silver or neutral copper particles), humectants, sequestering agents, texturing agents, or colorants. Exemplary additives include, but are not limited to, silicon dioxide (silica, SiO₂), titanium dioxide (titania, TiO₂), talc, silicates, aluminosilicates, butylated hydroxytoluene (BHT), sodium bicarbonate, and calcium carbonate, barium sulfate, mica, diatomite, wollastonite, calcium sulfate, zinc oxide, and carbon. Some additives, such as TiO₂ and SiO₂, may serve multiple functions.

The disinfectant composition is preferably stable to light (primarily UV light) and heat. If necessary, one or more additives (such as TiO₂) may be included specifically to confer UV resistance to the disinfectant composition. Other UV stabilizers include thiols, hindered amines (e.g., a derivative of tetramethylpiperidine), UV-absorbing particles (e.g., CdS, CdTe, or ZnS), or a combination thereof, for example.

When additives are included in the disinfectant composition, the particle size of the additives may vary. In some embodiments, the average particle size of an additive is selected from about 0.5 microns to about 100 microns. In various embodiments, the average particle size of an additive is about, at least about, or at most about 0.5, 0.6, 0.7, 0.8, 0.9, 1, 1.5, 2, 2.5, 3, 4, 5, 6, 7, 8, 9, 10, 15, 20, 25, 30, 40, 50, 60, 70, 80, 90, or 100 microns, including all intervening ranges. In certain embodiments, the average particle size of any additive is at least about 0.5 microns. Typically, the average particle size of any additive is greater than 0.1 micron, but nanoparticle additives with sizes less than 0.1 micron (100 nanometers) may optionally be employed.

Various methods of using disinfectant compositions may be employed, as will now be further described.

In some methods of using a disinfectant composition, the disinfectant composition—in solution, gel, spray, foam, dry, or other form—is applied to a food, a beverage, or water. When the disinfectant composition is applied to a food, the disinfectant composition may be applied to a food surface or may be impregnated within a food.

The step of application of the disinfectant composition to a food surface may include spraying, coating, casting, pouring, or other techniques. In some embodiments, a disinfectant composition is prepared and then dispensed (deposited) over an area of interest. Any known methods to deposit disinfectant compositions may be employed. Various coating techniques include, but are not limited to, spray coating, dip coating, doctor-blade coating, spin coating, air knife coating, curtain coating, single and multilayer slide coating, gap coating, knife-over-roll coating, metering rod (Meyer bar) coating, reverse roll coating, rotary screen coating, extrusion coating, casting, or printing. The disinfectant composition may be rapidly sprayed or cast in thin layers over large areas.

In some methods of using a disinfectant composition, the disinfectant composition—in solution, dry, or other form—is incorporated as a bulk component within a food. In these embodiments, the disinfectant composition is not solely at a surface but is also within the bulk region of the particular food material or object.

In this detailed description, reference has been made to multiple embodiments in which are shown by way of illustration specific exemplary embodiments of the invention. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein.

The embodiments, variations, and figures described above should provide an indication of the utility and versatility of the present invention. Other embodiments that do not provide all of the features and advantages set forth herein may also be utilized, without departing from the spirit and scope of the present invention. Such modifications and variations are considered to be within the scope of the invention defined by the claims. Furthermore, various aspects of the invention may be used in other applications than those for which they were specifically described herein.

### EXAMPLES

While the present invention is disclosed in reference to the preferred embodiments or examples above, it is to be understood that these embodiments or examples are intended for illustrative purposes, which shall not be treated as limitations to the present invention. It is contemplated that modifications and combinations will readily occur to those skilled in the art, which modifications and combinations will be within the spirit of the invention and the scope of the following claims.

### Materials and Instrument

The following materials were sourced in the Examples noted below: Ethylenediaminetetraacetic acid (EDTA), tartaric acid, lactic acid, citric acid, and acetic acid were sourced from Analab Fine Chemicals, Gujarat, India or Sigma Aldrich and used without further purification. The purity of these reagents was greater than 99%. Silver electrodes, copper electrodes, and zinc electrodes were sourced from Rochester Silver, Rochester, N.Y., Alpha Chemika, or Sigma Aldrich and cleaned before use. The electrodes were cleaned by wiping the electrodes with acetone followed by distilled water. Polyvinylpyrrolidone K-30 (PVP K-30) and polyvinylpyrrolidone K-90 (PVP K-90) was sourced from Alpha Chemika and used directly without further purification. Water utilized in these experiments was double distilled water.

The pH of the metal ion disinfectant composition was determined using a Systonic digital auto pH meter with Combination pH Electrode calibrated with a pH 7.0 buffer. The concentration of silver ions in the samples was determined by an inductively coupled plasma optical emission spectrometry (ICP-OES) method or potentiometric titration using 1 drop nitric acid and titrating with 100 ppm solution of sodium chloride. The presence of nanoparticles was determine using ultraviolet (UV)-visible spectroscopy.

### Example 1: General Procedure for the Preparation of Two Different Metal Ion Disinfectant Composition

Into a flask was added 100 mL of distilled water. Two electrodes were suspended and placed into the water. A 5V or a 12V DC battery was connected to the electrodes through a wire thus initiating the electrolysis. The electrolysis was conducted for a duration from 5 minutes to 30 minutes at room temperature. The battery was disconnected, and a second set of electrodes were suspended and placed into the reaction solution thus initiating the electrolysis. A 5V or a 12V DC battery was connected to these electrodes through a wire. After the electrolysis was complete, a magnetic stirring bar was added followed by the chelating agent. This mixture was stirred for 5 to 10 minutes until the chelating agent disappeared. Stirring was stopped and the solution stood at room temperature for 30 minutes to determine whether the multi-metal salt would precipitate. If the multi-metal salt precipitated, the multi-metal salt was isolated by filtration.

An ASTM E-2315 was conducted under guidelines of the AOAC (Association of Official Analytical Chemists). A pure culture of Escherichia coli (E. coli, ATCC 25922) was streaked on Soyabean Casein Digest Agar plates or MacConkey Agar with PES membrane filtration and allowed to incubate at 37° C. for up to 2 days. Following incubation, the surface of agar plate was scraped, and the growth suspension was adjusted to a concentration of 106 cfu/ml. Test and control substances were dispensed in identical volumes to sterile test tubes. Independently, test and control substances were inoculated with the test microorganism and mixed. Control suspensions were immediately plated to represent the concentration present at the start of the test or time zero and at the conclusion of each contact time; a volume of the liquid test solution was neutralized. Dilutions of the neutralized test solution were placed on to appropriate agar plates and incubation temperatures to determine the surviving microorganisms at the respective contact times and reductions of microorganisms were calculated by comparing initial microbial concentrations to surviving microbial concentrations. The samples showed greater than 99% reduction on exposure to Escherichia coli when exposed for just 15 seconds, thereby demonstrating instant killing activity of the composition as compared to the control. This data is presented in Table 1. Similar tests were conducted Pseudomonas aeruginosa (ATCC 9027) showing the same instant kill rate of the composition as compared to the control.

TABLE 1
Two Metal Multi-Metal Salt Compositions and ASTM E-2315 Evaluation

| | | | | | | | | Results |
|---|---|---|---|---|---|---|---|---|
| | | | | | | | Results | Pseudomonas |
| Experiment | Electrode | Time | Electrode | Time | Chelating agent | Voltage | E. Coli | aeruginosa |
| # | 1 | (min) | 2 | (min) | (g) | DC | (Reduction) | (Reduction) |
| 1 | Zn | 15 | Cu | 15 | EDTA (6 g) | 5 | 99.999999 | 99.999999 |
| 2 | Zn | 10 | Cu | 10 | EDTA (2 g) | 5 | 99.999999 | 99.999999 |
| 3 | Zn | 5 | Cu | 5 | EDTA (6 g) | 5 | 99.999999 | 99.999999 |
| 4 | Zn | 5 | Cu | 15 | EDTA (6 g) | 5 | 99.999999 | 99.999999 |
| 5 | Zn | 15 | Cu | 15 | EDTA (1 g) | 5 | 99.999999 | 99.0 |
| 6 | Zn | 15 | Cu | 15 | EDTA (6 g) | 12 | 99.999999 | 99.999999 |
| 8 | Zn | 15 | Cu | 15 | EDTA (6 g) | 12 | 99.999999 | 99.999999 |
| 9 | Zn | 5 | Cu | 5 | EDTA (1 g) | 12 | 99.9999 | 99.9999 |
| 10 | Zn | 25 | Cu | 25 | Citric Acid (2 g) | 5 | 99.999999 | 99.999999 |
| 11 | Zn | 15 | Cu | 15 | Citric Acid (2 g) | 5 | 99.999999 | X |
| 12 | Zn | 5 | Cu | 5 | Citric Acid (6 g) | 5 | 99.999999 | 99.999999 |
| 13 | Zn | 5 | Cu | 15 | Citric Acid (2 g) | 5 | 99.999999 | X |
| 14 | Zn | 5 | Cu | 5 | Citric Acid (6 g) | 12 | 99.999999 | 99.999999 |
| 15 | Zn | 5 | Cu | 5 | Citric Acid (1 g) | 12 | 99.999999 | 99.999999 |
| 16 | Zn | 25 | Cu | 25 | Tartaric Acid (2 g) | 5 | 99.999999 | 99.999999 |
| 17 | Zn | 15 | Cu | 15 | Tartaric Acid (2 g) | 5 | 99.999999 | X |
| 18 | Zn | 10 | Cu | 10 | Tartaric Acid (6 g)) | 5 | 99.999999 | 99.999999 |
| 19 | Zn | 5 | Cu | 5 | Tartaric Acid (6 g)) | 5 | 99.999999 | 99.999999 |
| 20 | Zn | 5 | Cu | 5 | Tartaric Acid (2 g)) | 5 | 99.999999 | 99.9 |
| 21 | Zn | 15 | Cu | 15 | Tartaric Acid (1 g) | 12 | 99.999999 | X |
| 22 | Zn | 25 | Cu | 25 | Lactic Acid (6 g) | 5 | 99.999999 | 99.999999 |
| 23 | Zn | 5 | Cu | 5 | Lactic Acid (2 g) | 5 | 99.999999 | 99.999999 |
| 24 | Ag | 15 | Cu | 15 | Citric Acid (2 g) | 5 | 99.99999 | 99.9 |
| 25 | Ag | 15 | Cu | 15 | Tartaric Acid (2 g) | 5 | 99.99999 | X |
| 26 | Zn | 15 | Ag | 15 | EDTA (6 g) | 5 | 99.999999 | X |
| 27 | Zn | 15 | Ag | 15 | EDTA (2 g) | 5 | 99.999999 | X |
| 28 | Zn | 15 | Ag | 15 | EDTA (1 g) | 5 | 99.999999 | X |
| 29 | Zn | 15 | Ag | 15 | Citric acid (2 g)) | 5 | 99.999999 | 99.0 |
| 30 | Zn | 15 | Ag | 15 | Tartaric Acid (2 g) | 5 | 99.999999 | 99.0 |

EDTA: ethylenediamine tetraacetic acid.

The data in the above table indicates that the metal ions prepared initially then complexed to a chelating agent are effective at reducing the pathogen level greater than 99% in less than 5 minutes.

### Example 2: Simultaneous Procedure for the Preparation of Two Different Metal Ion Disinfectant Composition

Into a flask was added 500 mL of distilled water. To this flask was added the chelating agent and a magnetic stirring bar. After the chelating agent dissolved, two zinc electrodes and two copper electrodes were suspended and placed into the water. Two 5V or a 12V DC batteries were connected to each set electrodes through wires thus initiating the electrolysis. The electrolysis was conducted for a duration of 15 minutes at room temperature. The battery was disconnected. The solution was stirred at room temperature for an additional 5 minutes and then evaluated for the ASTM-2315 as described in Example 1. The results of these experiments are shown in Table 2.

TABLE 2
Two Metal Multi-Metal Salt Compositions prepared by Simultaneous Electrolysis and ASTM E-2315 Evaluation

| | | | | | | | | Results | |
|---|---|---|---|---|---|---|---|---|---|
| | | | | | | | Results | Pseudomonas | Evaluation |
| Experiment | Chelating agent | Electrode | Electrode | Time | Water | Voltage | E. Coli | aeruginosa | Time |
| # | (g) | 1 | 2 | (min) | (mL) | DC | (Reduction) | (Reduction) | (min) |
| 31 | Citric Acid (5 g) | Zn | Cu | 15 | 500 | 5 V | X | 55 | 10 |
| 32 | Citric Acid (10 g) | Zn | Cu | 15 | 500 | 5 V | X | 84 | 10 |
| 33 | Tartaric Acid (5 g)) | Zn | Cu | 15 | 500 | 5 V | X | X | 5 |
| 34 | Tartaric Acid (10 g)) | Zn | Cu | 15 | 500 | 5 V | X | X | 5 |
| 35 | Lactic Acid (5 g)) | Zn | Cu | 15 | 500 | 5 V | X | X | 5 |
| 36 | Lactic Acid (10 g)) | Zn | Cu | 15 | 500 | 5 V | X | X | 5 |

The examples demonstrates that the disinfectant composition can be prepared. With inclusion of the chelating agent before the electrolysis, the chelating agent did not fully complex the multi-metals and did not produce a disinfectant composition with a 99% or greater kill rate on a number of pathogens after 5 minutes.

### Example 3: General Procedure for the Preparation of Three Different Metal Ion Disinfectant Composition

Into a flask was added 100 mL of distilled water. Two electrodes were suspended and placed into the water. A 5V or a 12V DC battery was connected to the electrodes through a wire thus initiating the electrolysis. The electrolysis was conducted for a duration from 5 minutes to 30 minutes at room temperature. The battery was disconnected, and a second set of electrodes were suspended and placed into the reaction solution thus initiating the electrolysis. A 5V or a 12V DC battery was connected to these electrodes through a wire. The battery was disconnected, and a third set of electrodes were suspended and placed into the reaction solution thus initiating the electrolysis. After the electrolysis was complete, a magnetic stirring bar was added followed by the chelating agent. This mixture was stirred for 5 to 10 minutes until the chelating agent disappeared. Stirring was stopped and the solution stood at room temperature for 30 minutes to determine whether the multi-metal salt would precipitate. If the multi-metal salt precipitated, the multi-metal salt was isolated by filtration. Table 3 shows the conditions and ASTM-2315 Evaluation of the Disinfectant Composition.

TABLE 3
Three Metal Multi-Metal Salt Compositions and ASTM E-2315 Evaluation

| | | | | | | | | | Results | Results |
|---|---|---|---|---|---|---|---|---|---|---|
| Experiment | Electrode | Time | Electrode | Time | Electrode | Time | Voltage | Chelating Agent | E. Coli | pseudomonas |
| # | 1 | (min) | 2 | (min | 3 | (min) | DC | (g) | (% Reduction) | (% Reduction) |
| 37 | Zn | 25 | Cu | 25 | Silver | 25 | 5 | EDTA (6 g) | 99.999999 | X |
| 38 | Zn | 25 | Cu | 25 | Silver | 25 | 5 | EDTA (3 g) | 99.999999 | 99.9 |
| 39 | Zn | 15 | Cu | 15 | Silver | 15 | 5 | EDTA (6 g) | 99.999999 | 99.999999 |
| 40 | Zn | 15 | Cu | 15 | Silver | 15 | 5 | EDTA (6 g) | 99.999999 | 99.999999 |
| 41 | Zn | 15 | Cu | 15 | Silver | 15 | 5 | EDTA (3 g) | 99.999999 | 99.999999 |
| 42 | Zn | 10 | Cu | 10 | Silver | 10 | 5 | EDTA (3 g) | 99.999999 | 99.999999 |
| 43 | Zn | 10 | Cu | 10 | Silver | 10 | 5 | EDTA (1 g) | 99.99999 | 99.99999 |
| 44 | Zn | 5 | Cu | 5 | Silver | 5 | 5 | EDTA (6 g) | 99.999999 | 99.999999 |
| 45 | Zn | 25 | Cu | 25 | Silver | 25 | 5 | Citric Acid (3 g) | 99.999999 | 99.999999 |
| 46 | Zn | 15 | Cu | 15 | Silver | 15 | 5 | Citric Acid (6 g) | 99.999999 | 99.999999 |
| 47 | Zn | 15 | Cu | 15 | Silver | 15 | 5 | Citric Acid (3 g) | 99.999999 | 99.999999 |
| 48 | Zn | 15 | Cu | 15 | Silver | 15 | 5 | Citric Acid (1 g) | 99.999999 | 99.999999 |
| 49 | Zn | 10 | Cu | 10 | Silver | 10 | 5 | Citric Acid (3 g) | 99.999999 | 99.99 |
| 50 | Zn | 5 | Cu | 5 | Silver | 5 | 5 | Citric Acid (3 g) | 99.999999 | 99.999999 |
| 51 | Zn | 25 | Cu | 25 | Silver | 25 | 5 | Tartaric Acid (6 g) | 99.999999 | 99.999999 |
| 52 | Zn | 10 | Cu | 10 | Silver | 10 | 5 | Tartaric Acid (3 g) | 99.999999 | 99.999999 |
| 53 | Zn | 5 | Cu | 5 | Silver | 5 | 5 | Tartaric Acid (6 g) | 99.999999 | 99.999999 |
| 54 | Zn | 10 | Cu | 10 | Silver | 10 | 5 | Tartaric Acid (1 g) | 99.999999 | 99.9 |
| 55 | Zn | 15 | Cu | 15 | Silver | 15 | 12 | Tartaric Acid (1 g) | 99.999999 | 99.9 |
| 56 | Zn | 5 | Cu | 5 | Silver | 5 | 12 | Tartaric Acid (3 g) | 99.999999 | 99.9 |
| 57 | Zn | 5 | Cu | 5 | Silver | 5 | 12 | Tartaric Acid 1 g) | 99.999999 | |
| 58 | Zn | 25 | Cu | 25 | Silver | 25 | 5 | Lactic Acid (3 g) | 99.999999 | 99.9 |
| 59 | Zn | 15 | Cu | 15 | Silver | 15 | 5 | Lactic Acid (3 g) | 99.999999 | 99.9 |
| 60 | Zn | 10 | Cu | 10 | Silver | 10 | 5 | Lactic Acid (6 g) | 99.999999 | 99.999999 |
| 61 | Zn | 10 | Cu | 10 | Silver | 10 | 5 | Lactic Acid (1 g) | 99.999999 | 99.99999 |
| 62 | Zn | 5 | Cu | 5 | Silver | 5 | 5 | Lactic Acid (1 g) | 99.999999 | 99.999 |
| 63 | Zn | 15 | Cu | 15 | Silver | 15 | 12 | Lactic Acid (1 g) | 99.999999 | 99.999999 |
| 64 | Zn | 5 | Cu | 5 | Silver | 5 | 12 | Lactic Acid (1 g) | 99.999999 | |

The data in the above table indicates that the metal ions prepared initially then complexed to a chelating agent are effective at reducing the pathogen level greater than 99% in less than 5 minutes.

### Example 4: Preparation of Two Different Metal Ion Disinfectant Composition Comprising a Multi-Metal Salt and a Polymer

Into a flask was added 100 mL of distilled water. Two electrodes were suspended and placed into the water. A 12V DC battery was connected to the electrodes through a wire thus initiating the electrolysis. The electrolysis was conducted for a duration from 5 minutes to 30 minutes at room temperature. The battery was disconnected, and a second set of electrodes were suspended and placed into the reaction solution thus initiating the electrolysis. A 12V DC battery was connected to these electrodes through a wire. After the electrolysis was complete, a magnetic stirring bar was added followed by the chelating agent. This mixture was stirred for 5 to 10 minutes until the chelating agent dissolved. Then, a hydrophilic polymer, PVP K-90, was added in portions. After stirring for 10 minutes, the disinfectant composition was prepared as a colorless solution. Table 4, shown below, indicates the amounts used in this Example and the ASTM-2315 evaluation results.

TABLE 4
Two Metal Multi-Metal Salt and Polymer Composition and ASTM E-2315 Evaluation

| Experiment | Electrode | | Electrode | | Electrode | | DC | Chelating | polymer | Water |
|---|---|---|---|---|---|---|---|---|---|---|
| # | 1 | Duration | 2 | Duration | 3 | Duration 3 | Voltage | Agent | (g) | (mL) |
| 65 | Zn | 15 | Cu | 15 | X | X | 12 | Tartaric Acid (1) | PVP K90 (2.0 g) | 100 |

| | Results | Results |
|---|---|---|
| Experiment | E. Coli | Pseudomonas aeruginosa |
| # | (% Reduction) | (% Reduction) |
| 65 | 99.999999 | 99.999999 |

### Example 5: Preparation of Three Different Metal Ion Disinfectant Composition Comprising a Three Multi-Metal Salt and a Polymer

Into a flask was added 100 mL of distilled water and the chelating agent. Two electrodes were suspended and placed into the water. A 12V DC battery was connected to the electrodes through a wire thus initiating the electrolysis. The electrolysis was conducted for a duration from 5 minutes to 30 minutes at room temperature. The battery was disconnected, and a second set of electrodes were suspended and placed into the reaction solution thus initiating the electrolysis. A 12V DC battery was connected to these electrodes through a wire. The battery was disconnected, and a third set of electrodes were suspended and placed into the reaction solution thus initiating the electrolysis. After the electrolysis was complete, a magnetic stirring bar was added followed by the hydrophilic polymer. This mixture was stirred for 5 to 10 minutes until a clear solution appeared. Stirring was stopped and the solution stood at room temperature for 30 minutes to determine whether the multi-metal salt would precipitate. If the multi-metal salt precipitated, the multi-metal salt was isolated by filtration. Table 5 shows the conditions and ASTM-2315 Evaluation of the Disinfectant Composition.

TABLE 5
Three Metal Multi-Metal Salt and Polymer Composition and ASTM E-2315 Evaluation

| Experiment | Electrode | | Electrode | | Electrode | Duration | DC | Chelating | polymer | Water |
|---|---|---|---|---|---|---|---|---|---|---|
| # | 1 | Duration | 2 | Duration | 3 | 3 | Voltage | Agent | (g) | (mL) |
| 66 | Zn | 15 | Cu | 15 | Ag | 15 | 12 | Lactic Acid (1 g) | PVP K90 (2.0 g) | 100 |
| 67 | Zn | 15 | Cu | 15 | Ag | 15 | 12 | Tartaric Acid) | PVP K90 (2.0 g) | 100 |

| | Results | Results |
|---|---|---|
| Experiment | E. Coli | Pseudomonas aeruginosa |
| # | (Reduction) | (% Reduction) |
| 66 | 99.999999 | 99.999999 |
| 67 | 99.999999 | 99.999999 |

The addition of the chelating agent before electrolysis did not affect the kill rate of the disinfectant composition.

### Example 6: Preparation of Three Different Metal Ion Disinfectant Composition Comprising a Three Multi-Metal Salt and a Polymer

Into a flask was added 100 mL of distilled water. Two electrodes were suspended and placed into the water. A 12V DC battery was connected to the electrodes through a wire thus initiating the electrolysis. The electrolysis was conducted for a duration from 5 minutes to 30 minutes at room temperature. The battery was disconnected, and a second set of electrodes were suspended and placed into the reaction solution thus initiating the electrolysis. A 12V DC battery was connected to these electrodes through a wire. The battery was disconnected, and a third set of electrodes were suspended and placed into the reaction solution thus initiating the electrolysis. After the electrolysis was complete, a magnetic stirring bar was added followed by the chelating agent. This mixture was stirred for 5 to 10 minutes until a clear solution appeared. Then, the hydrophilic polymer was added. This mixture was stirred for 5 to 10 minutes until a clear solution appeared. Stirring was stopped and the solution stood at room temperature for 30 minutes to determine whether the multi-metal salt would precipitate. If the multi-metal salt precipitated, the multi-metal salt was isolated by filtration. Table 6 shows the conditions and ASTM-2315 Evaluation of the Disinfectant Composition.

TABLE 6
Three Metal Multi-Metal Salt and Polymer Composition and ASTM E-2315 Evaluation

| Experiment | Electrode | | Electrode | | Electrode | Duration | DC | Chelating | polymer | Water |
|---|---|---|---|---|---|---|---|---|---|---|
| # | 1 | Duration | 2 | Duration | 3 | 3 | Voltage | Agent | (g) | (mL) |
| 68 | Zn | 15 | Cu | 15 | Ag | 15 | 12 | Tartaric Acid (1 g) | PVP K90 (2.0 g) | 100 |

| | Results | Results |
|---|---|---|
| Experiment | E. Coli | Pseudomonas aeruginosa |
| # | (Reduction) | (Reduction) |
| 68 | 99.999999 | 99.999999 |

This example shows that the addition of the chelating agent after the electrolysis did not affect the kill rate of the disinfectant composition.

What is claimed is:

1. A disinfectant composition comprising:
a multi-metal salt, wherein said multi-metal salt includes at least two different metal salts; and
a chelating agent;
wherein the disinfectant composition does not contain nanoparticles.

2. The disinfectant composition of claim 1, wherein the composition further comprises water,

3. The disinfectant composition of claim 1, wherein the composition further comprises a surfactant, a polymer, an additive or combination thereof.

4. The disinfectant composition of claim 1, wherein the at least two metal salts are selected from a group consisting of a silver salt, a copper salt, a zinc salt, a gold salt, a cobalt salt, a nickel salt, a zirconium salt, a molybdenum salt, a palladium salt, and combinations thereof.

5. The disinfectant composition of claim 4, wherein the at least two metal salts are a silver salt, a copper salt, a zinc salt, or a combination thereof.

6. The disinfectant composition of claim 5, wherein the at least two metal salts are a silver salt and a copper salt.

7. The disinfectant composition of claim 5, wherein the at least two metal salts are a silver salt and a zinc salt.

8. The disinfectant composition of claim 5, wherein the at least two metal salts are a copper salt and a zinc salt.

9. The disinfectant composition of claim 5, wherein the at least two metal salts are a copper salt, a zinc salt, and a silver salt.

10. The disinfectant composition of claim 1, wherein the multi-metal salt has a concentration from about 0.001 wt % to about 50 wt %.

11. The disinfectant composition of claim 1, wherein the concentrations of each metal salt may be the same or different.

12. The disinfectant composition of claim 1, wherein the chelating agent is selected from a group consisting of citric acid, lactic acid, acetic acid, ethylenediaminetetraacetic acid, or a combination thereof.

13. The disinfectant composition of claim 1, wherein the chelating agent to the water has a weight to volume ratio from about 0.1:100.0 to about 50.0:100.0.

14. The disinfectant composition of claim 1, wherein the chelating agent to the water has a weight to volume ratio from about 0.5:100.0 to about 10.0:100.0.

15. The disinfectant composition of claim 1, wherein the disinfectant composition has a pH of about 6 to about 8.

16. The disinfectant composition of claim 1, wherein the composition is non-toxic and non-corrosive.

17. The disinfectant composition of claim 1, wherein the composition is light and heat stable.

18. The disinfectant composition of claim 1, wherein the at least one hydrophilic polymer prevents oxidation and/or moisture of the at least one metal ion.

19. The disinfectant composition of claim 1, wherein the maintains contact on a variety of surfaces.

20. The composition of claim 1, wherein the disinfectant composition exhibits antimicrobial properties, antibacterial properties, antifungal properties, antiviral properties, or a combination thereof against a variety of pathogens.

21. The disinfectant composition of claim 1, wherein the composition achieves a greater than a 99% kill rate on a variety of surfaces in less than 5 minutes.

22. The disinfectant composition of claim 1, wherein the composition remains efficacious and/or maintains pathogenic sterility for up to 60 days on a variety of surfaces.

23. A method of disinfecting an article comprising contacting the article with the disinfecting composition of claim 1.

24. The method of claim 22, wherein the method kills greater than 99% of pathogens on the article.
